SEQUENCE LISTING (33 sequences)

SEQ ID NO: 1 - protein, 96 aa, synthetic construct
MKKYVIEEELSDEELLEKFKEIVELMKEEPVDITVTGAHALEIFRAVTELAKLLGVKVNIDVTLTESSAKYEPLEKKMLEYVRETVEAEKKKEELA

SEQ ID NO: 2 - protein, 91 aa, synthetic construct
KSVVYELTYEDKSGNKVKVTVTYDEETGEITLTQERTSKRVVNGKLVEVKETFLWRLSSSGTAEEALATIKKAYQRGAKTPVKVSFVKEIS

SEQ ID NO: 3 - protein, 75 aa, synthetic construct
MMKNVDVDIKYDKDGTIKVTIKSEDTTIEIEEKVKTKEEREAVLDIVFEVMRRLLKGEDIEEIKKKMEEELKKIK

SEQ ID NO: 4 - protein, 202 aa, synthetic construct
MKKYVIEEELSDEELLEKFKEIVELMKEEPVDITVTGAHALEIFRAVTELAKLLGVKVNIDVTLTESSAKYEPLEKKMLEYVRETVEAEKKKEELAGGGGSGGGGSGGGGSKSVVYELTYEDKSGNKVKVTVTYDEETGEITLTQERTSKRVVNGKLVEVKETFLWRLSSSGTAEEALATIKKAYQRGAKTPVKVSFVKEIS

SEQ ID NO: 5 - protein, 202 aa, synthetic construct
KSVVYELTYEDKSGNKVKVTVTYDEETGEITLTQERTSKRVVNGKLVEVKETFLWRLSSSGTAEEALATIKKAYQRGAKTPVKVSFVKEISGGGGSGGGGSGGGGSMKKYVIEEELSDEELLEKFKEIVELMKEEPVDITVTGAHALEIFRAVTELAKLLGVKVNIDVTLTESSAKYEPLEKKMLEYVRETVEAEKKKEELA

SEQ ID NO: 6 - protein, 56 aa, synthetic construct
SEKEEELKKLIEEAIKLEKEGKKEEAKKKLEEALELAKELGYDATAEAVQQKLDKL

SEQ ID NO: 7 - DNA, 288 bp, synthetic construct
  source 1..288: mol_type = other DNA
atgaaaaaatacgtgattgaagaagaactgtcggatgaagaactgctggaaaaattcaaagaaattgtggaactgatgaaagaagaaccggtggatattaccgtgaccggcgcgcatgccctggaaatttttcgcgccgtgacggaactggccaaactgctgggcgtgaaagtgaatattgatgtgaccctgaccgaaagcagcgccaaatacgaaccgctggaaaaaaaaatgctggaatatgtgcgcgaaaccgtggaagcggaaaaaaaaaaagaagaactggcg

SEQ ID NO: 8 - DNA, 273 bp, synthetic construct
  source 1..273: mol_type = other DNA
aaaagcgtggtttatgaactgacctatgaagataaaagcggcaacaaagtgaaagtgaccgtgacctatgatgaagaaaccggtgaaattaccctgacccaggaacgtaccagcaaacgcgtggtgaacggcaaactggtggaggtgaaagaaacctttctgtggcgtctgagcagcagcggcaccgccgaagaagcgctggccaccattaaaaaagcgtaccagcgcggtgcgaaaaccccggtgaaagtgagctttgtgaaagaaattagc

SEQ ID NO: 9 - DNA, 225 bp, synthetic construct
  source 1..225: mol_type = other DNA
atgatgaaaaacgtggatgtggacattaagtacgataaagatggcaccatcaaagtgaccattaaaagcgaagataccaccatcgaaatcgaagaaaaagtcaaaaccaaggaagaacgcgaagcggtgctggatattgtgttcgaagtgatgcgccgtctgctgaagggcgaagatatcgaggaaattaaaaaaaaaatggaagaagaactgaaaaaaattaaa

SEQ ID NO: 10 - DNA, 606 bp, synthetic construct
  source 1..606: mol_type = other DNA
atgaaaaaatacgtgattgaagaagaactgtcggatgaagaactgctggaaaaattcaaagaaattgtggaactgatgaaagaagaaccggtggatattaccgtgaccggcgcgcatgccctggaaatttttcgcgccgtgacggaactggccaaactgctgggcgtgaaagtgaatattgatgtgaccctgaccgaaagcagcgccaaatacgaaccgctggaaaaaaaaatgctggaatatgtgcgcgaaaccgtggaagcggaaaaaaaaaaagaagaactggcgggcggcggcggcagcggtggtggtggtagtggcggcggcggcagcaaaagcgtggtttatgaactgacctatgaagataaaagcggcaacaaagtgaaagtgaccgtgacctatgatgaagaaaccggtgaaattaccctgacccaggaacgtaccagcaaacgcgtggtgaacggcaaactggtggaggtgaaagaaacctttctgtggcgtctgagcagcagcggcaccgccgaagaagcgctggccaccattaaaaaagcgtaccagcgcggtgcgaaaaccccggtgaaagtgagctttgtgaaagaaattagc

SEQ ID NO: 11 - DNA, 606 bp, synthetic construct
  source 1..606: mol_type = other DNA
aaaagcgtggtttatgaactgacctatgaagataaaagcggcaacaaagtgaaagtgaccgtgacctatgatgaagaaaccggtgaaattaccctgacccaggaacgtaccagcaaacgcgtggtgaacggcaaactggtggaggtgaaagaaacctttctgtggcgtctgagcagcagcggcaccgccgaagaagcgctggccaccattaaaaaagcgtaccagcgcggtgcgaaaaccccggtgaaagtgagctttgtgaaagaaattagcggcggcggcggcagcggtggtggtggtagtggcggcggcggcagcatgaaaaaatacgtgattgaagaagaactgtcggatgaagaactgctggaaaaattcaaagaaattgtggaactgatgaaagaagaaccggtggatattaccgtgaccggcgcgcatgccctggaaatttttcgcgccgtgacggaactggccaaactgctgggcgtgaaagtgaatattgatgtgaccctgaccgaaagcagcgccaaatacgaaccgctggaaaaaaaaatgctggaatatgtgcgcgaaaccgtggaagcggaaaaaaaaaaagaagaactggcg

SEQ ID NO: 12 - DNA, 168 bp, synthetic construct
  source 1..168: mol_type = other DNA
agcgaaaaagaagaagaactgaaaaaactgattgaagaagcgattaaactggaaaaagaaggcaaaaaagaagaagcgaaaaaaaaactggaagaagcgctggaactggcgaaagaactgggctacgatgccaccgcggaagcggtgcagcagaaactggataaactg

SEQ ID NO: 13 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccgaatttgaaattcgtcataatgtggaagatggcagcgtgcagctggcggatcattatcagcagaataccccgattggcgatggcccagtgctgctgccggatgaccactatctgagcaccgaaagcgtgctgagcaaagatccgaatgaagatcgtgatcatatggtcctgctggaatttgtgaccgcggcaggcattgatctgggcatggatgaactgtataaa

SEQ ID NO: 14 - DNA, 96 bp, synthetic construct
  source 1..96: mol_type = other DNA
ggcggcggcggcagcggtggtggtggtagtggcggcggcggcagctacccgtatgatgttccggattacgctttggaggttttgttccagggtcca

SEQ ID NO: 15 - DNA, 51 bp, synthetic construct
  source 1..51: mol_type = other DNA
tacccgtatgatgttccggattacgctttggaggttttgttccagggtcca

SEQ ID NO: 16 - protein, 234 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 17 - protein, 234 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRR

SEQ ID NO: 18 - protein, 16 aa, synthetic construct
GGGGSYPYDVPDYAGS

SEQ ID NO: 19 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcagccaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 20 - DNA, 702 bp, synthetic construct
  source 1..702: mol_type = other DNA
gcttcagcagcacgcttgacaatgatgtgggaggaggtcacatgccctatctgcctggaccccttcgtggagcctgtgagcatcgagtgtggccacagcttctgccaggaatgcatctctcaggttgggaaaggtgggggcagcgtctgtcctgtgtgccggcagcgctttctgctcaagaatctccggcccaatcgacagctagccaacatggtgaacaaccttaaagaaatcgaacaggaggccagagagggcacacagggggaacggtgtgcagtgcatggagagagacttcacctgttctgtgagaaagatgggaaggccctttgctgggtatgtgcccagtctcggaaacaccgtgaccacgccatggtccctcttgaggaggctgcacaggagtaccaggagaagctccaggtggcattaggggaactgagaagaaagcaggagttggctgagaagttggaagtggaaattgcaataaagagagcagactggaagaaaacagtggaaacacagaaatctaggattcacgcagagtttgtgcagcaaaaaaacttcctggttgaagaagaacagaggcagctgcaggagctggagaaggatgagagggagcagctgagaatcctgggggagaaagaggccaagctggcccagcagagccaggccctacaggagctcatctcagagctagatcgaagg

SEQ ID NO: 21 - DNA, 48 bp, synthetic construct
  source 1..48: mol_type = other DNA
ggcggaggtggcagctatccgtatgatgttccggattatgcaggaagc

SEQ ID NO: 22 - protein, 346 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMKKYVIEEELSDEELLEKFKEIVELMKEEPVDITVTGAHALEIFRAVTELAKLLGVKVNIDVTLTESSAKYEPLEKKMLEYVRETVEAEKKKEELA

SEQ ID NO: 23 - protein, 341 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSKSVVYELTYEDKSGNKVKVTVTYDEETGEITLTQERTSKRVVNGKLVEVKETFLWRLSSSGTAEEALATIKKAYQRGAKTPVKVSFVKEIS

SEQ ID NO: 24 - protein, 325 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMMKNVDVDIKYDKDGTIKVTIKSEDTTIEIEEKVKTKEEREAVLDIVFEVMRRLLKGEDIEEIKKKMEEELKKIK

SEQ ID NO: 25 - protein, 452 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMKKYVIEEELSDEELLEKFKEIVELMKEEPVDITVTGAHALEIFRAVTELAKLLGVKVNIDVTLTESSAKYEPLEKKMLEYVRETVEAEKKKEELAGGGGSGGGGSGGGGSKSVVYELTYEDKSGNKVKVTVTYDEETGEITLTQERTSKRVVNGKLVEVKETFLWRLSSSGTAEEALATIKKAYQRGAKTPVKVSFVKEIS

SEQ ID NO: 26 - protein, 452 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSKSVVYELTYEDKSGNKVKVTVTYDEETGEITLTQERTSKRVVNGKLVEVKETFLWRLSSSGTAEEALATIKKAYQRGAKTPVKVSFVKEISGGGGSGGGGSGGGGSMKKYVIEEELSDEELLEKFKEIVELMKEEPVDITVTGAHALEIFRAVTELAKLLGVKVNIDVTLTESSAKYEPLEKKMLEYVRETVEAEKKKEELA

SEQ ID NO: 27 - protein, 306 aa, synthetic construct
ASAARLTMMWEEVTCPICLDPFVEPVSIECGHSFCQECISQVGKGGGSVCPVCRQRFLLKNLRPNRQLANMVNNLKEISQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSSEKEEELKKLIEEAIKLEKEGKKEEAKKKLEEALELAKELGYDATAEAVQQKLDKL

SEQ ID NO: 28 - protein, 346 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMKKYVIEEELSDEELLEKFKEIVELMKEEPVDITVTGAHALEIFRAVTELAKLLGVKVNIDVTLTESSAKYEPLEKKMLEYVRETVEAEKKKEELA

SEQ ID NO: 29 - protein, 452 aa, synthetic construct
ASAARLTMMWEEVTCPICLDPFVEPVSIECGHSFCQECISQVGKGGGSVCPVCRQRFLLKNLRPNRQLANMVNNLKEIEQEAREGTQGERCAVHGERLHLFCEKDGKALCWVCAQSRKHRDHAMVPLEEAAQEYQEKLQVALGELRRKQELAEKLEVEIAIKRADWKKTVETQKSRIHAEFVQQKNFLVEEEQRQLQELEKDEREQLRILGEKEAKLAQQSQALQELISELDRRGGGGSYPYDVPDYAGSMKKYVIEEELSDEELLEKFKEIVELMKEEPVDITVTGAHALEIFRAVTELAKLLGVKVNIDVTLTESSAKYEPLEKKMLEYVRETVEAEKKKEELAGGGGSGGGGSGGGGSKSVVYELTYEDKSGNKVKVTVTYDEETGEITLTQERTSKRVVNGKLVEVKETFLWRLSSSGTAEEALATIKKAYQRGAKTPVKVSFVKEIS

SEQ ID NO: 30 - protein, 275 aa, synthetic construct
SEKEEELKKLIEEAIKLEKEGKKEEAKKKLEEALELAKELGYDATAEAVQQKLDKLGSGSSLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 553 aa, synthetic construct
MHHHHHHSEKEEELKKLIEEAIKLEKEGKKEEAKKKLEEALELAKELGYDATAEAVQQKLDKLGGGGSGGGGSGGGGSYPYDVPDYALEVLFQGPMVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKAEFEIRHNVEDGSVQLADHYQQNTPIGDGPVLLPDDHYLSTESVLSKDPNEDRDHMVLLEFVTAAGIDLGMDELYKGSGSSLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 825 bp, synthetic construct
  source 1..825: mol_type = other DNA
agcgaaaaagaagaagaactgaaaaaactgattgaagaagcgattaaactggaaaaagaaggcaaaaaagaagaagcgaaaaaaaaactggaagaagcgctggaactggcgaaagaactgggctacgatgccaccgcggaagcggtgcagcagaaactggataaactgggatccgggtcctctttgttgggtggtccatccgtcttcttgtttccacctaagccaaaggacacccttatgatctccagaaccccagaagtcacctgcgttgtcgttgacgtttcccatgaggaccctgaggtcaagttcaactggtacgttgacggtgttgaggtccacaacgccaagaccaagccaagagaggagcagtacaactctacctaccgtgtcgtctccgtcttgaccgttttgcaccaggactggttgaacggtaaggagtacaaatgtaaggtttctaacaaggccttgccagccccaatcgagaagaccatctccaaggctaagggtcagcctagagaaccacaggtctacaccttgccaccttctagagaggagatgaccaagaaccaggtctccttgacctgtttggtcaagggattctacccatctgacatcgccgttgagtgggagtctaacggacagccagagaacaactacaagaccaccccacctgttttggactctgatggttctttttttttgtactctaagttgaccgttgacaagtcccgttggcaacagggtaacgttttctcctgctccgtcatgcacgaggccttgcataaccactacactcagaaatctttgtctttgtctccaggtaag

SEQ ID NO: 33 - DNA, 1659 bp, synthetic construct
  source 1..1659: mol_type = other DNA
atgcaccatcatcatcatcatagcgaaaaagaagaagaactgaaaaaactgattgaagaagcgattaaactggaaaaagaaggcaaaaaagaagaagcgaaaaaaaaactggaagaagcgctggaactggcgaaagaactgggctacgatgccaccgcggaagcggtgcagcagaaactggataaactgggcggcggcggcagcggtggtggtggtagtggcggcggcggcagctacccgtatgatgttccggattacgctttggaggttttgttccagggtccaatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccgaatttgaaattcgtcataatgtggaagatggcagcgtgcagctggcggatcattatcagcagaataccccgattggcgatggcccagtgctgctgccggatgaccactatctgagcaccgaaagcgtgctgagcaaagatccgaatgaagatcgtgatcatatggtcctgctggaatttgtgaccgcggcaggcattgatctgggcatggatgaactgtataaaggatccgggtcctctttgttgggtggtccatccgtcttcttgtttccacctaagccaaaggacacccttatgatctccagaaccccagaagtcacctgcgttgtcgttgacgtttcccatgaggaccctgaggtcaagttcaactggtacgttgacggtgttgaggtccacaacgccaagaccaagccaagagaggagcagtacaactctacctaccgtgtcgtctccgtcttgaccgttttgcaccaggactggttgaacggtaaggagtacaaatgtaaggtttctaacaaggccttgccagccccaatcgagaagaccatctccaaggctaagggtcagcctagagaaccacaggtctacaccttgccaccttctagagaggagatgaccaagaaccaggtctccttgacctgtttggtcaagggattctacccatctgacatcgccgttgagtgggagtctaacggacagccagagaacaactacaagaccaccccacctgttttggactctgatggttctttttttttgtactctaagttgaccgttgacaagtcccgttggcaacagggtaacgttttctcctgctccgtcatgcacgaggccttgcataaccactacactcagaaatctttgtctttgtctccaggtaag

(19) United States
(12) Patent Application Publication
(10) Pub. No.: US 2026/0285916 A1
(43) Pub. Date: Sep. 24, 2026

(54) BINDER AND FUSION PROTEIN TARGETING PCNA, AND USES THEREOF

(71) Applicant: Hubei University, Wuhan (CN)

(72) Inventors: Lixin MA, Wuhan (CN); Fei WANG, Wuhan (CN); Shuai ZHAO, Wuhan (CN); Yang WANG, Wuhan (CN); Fang YU, Wuhan (CN); Jingwei ZENG, Wuhan (CN); Wanping CHEN, Wuhan (CN)

(73) Assignee: Hubei University, Wuhan (CN)

(21) Appl. No.: 19/571,565

(22) Filed: Mar. 19, 2026

(30) Foreign Application Priority Data
Mar. 19, 2025 (CN) 202510328164.5

(51) Int. Cl. C07K 14/245; A61K 38/00; C12N 15/70; C12N 15/85

(52) U.S. Cl. CPC C07K 14/245; C12N 15/70; C12N 15/85; A61K 38/00; C07K 2319/02; C07K 2319/95

(57) ABSTRACT

The present disclosure provides a binder that specifically targets the PCNA protein, a fusion protein comprising the same, and uses thereof, which belong to the field of biotechnology. In the present disclosure, artificial intelligence technology is employed to design binders that specifically target PCNA. The resulting binders exhibit good specificity and high affinity, and are capable of efficiently binding to the PCNA antigen. Further, fusion proteins are prepared by fusing the aforementioned binders to an RBCC domain or a mutant thereof, or by directly fusing the aforementioned binders to an Fc fragment. The fusion proteins can specifically bind to the target protein via their binder domain, thereby initiating a protein degradation pathway to achieve targeted degradation of the PCNA protein, and they can also activate intracellular p53, inducing its phosphorylation to prevent ubiquitination-mediated degradation by ubiquitin ligases such as MDM2, thus giving the present disclosure substantial clinical application value.

### CROSS-REFERENCE TO RELATED APPLICATIONS

The application claims priority to Chinese patent application No. 202510328164.5, filed on Mar. 19, 2025, with the entire contents incorporated herein by reference.

### SEQUENCE LISTING

The sequence listing xml file submitted herewith, named “TYUS2604_Sequence_Listing.xml”, created on Mar. 10, 2026, and having a file size of 41,370 bytes, is incorporated by reference herein.

### TECHNICAL FIELD

The present disclosure relates to the field of biotechnology, and in particular to a binder specifically targeting PCNA, a fusion protein comprising the same, and uses thereof.

### BACKGROUND

The Proliferating Cell Nuclear Antigen (PCNA) gene encodes a nuclear protein that is primarily involved in DNA replication and repair processes. PCNA forms a trimeric ring structure that slides along DNA strands and acts as a loading factor for DNA polymerases, ensuring the high efficiency and accuracy of DNA replication. PCNA plays a pivotal role in cell proliferation, and its expression level is closely correlated with the cellular proliferation rate. During the initiation and elongation phases of DNA replication, PCNA functions as a sliding clamp which interacts with various replication and repair proteins to ensure the high efficiency and accuracy of DNA replication. PCNA is also involved in multiple DNA repair pathways, including base excision repair (BER), nucleotide excision repair (NER), and mismatch repair (MMR). These repair mechanisms are essential for maintaining genomic stability and preventing the accumulation of mutations.

PCNA is highly expressed in various cancers, and its expression level is positively correlated with the proliferation rate and malignancy of tumor cells. Owing to its pivotal role in DNA replication and repair, aberrant expression or dysfunction of PCNA can induce genomic instability, thereby promoting the development and progression of cancer. Therefore, PCNA has become an important therapeutic and research target for cancer.

Existing PCNA inhibitors primarily inhibit DNA replication and repair by impeding the interaction of PCNA with DNA polymerases or other repair proteins. However, research on PCNA degraders remains relatively limited. Currently available PCNA inhibitors exhibit limited efficacy and may be accompanied by a high risk of side effects. Thus, there is a critical need to develop more efficient and specific PCNA-targeted degradation technologies.

Binders are a class of small molecules, peptides or proteins with high specificity and affinity that can specifically bind to target proteins. Therefore, binders demonstrate broad application potential in biomedical research and therapeutic development.

Targeted Protein Degradation (TPD) technology utilizes the intracellular ubiquitin-proteasome system and lysosomal degradation system to achieve specific and efficient degradation of disease-associated proteins, thereby attaining therapeutic effects for diseases. Compared with traditional small-molecule inhibitors, TPD offers multiple advantages: it is event-driven, requires a low therapeutic dose and exerts its effects at only a catalytic dose; moreover, it can target some proteins that were previously considered undruggable.

TRIM away is an emerging TPD technology generated by fusing a binder to TRIM21, a member of the tripartite motif-containing (TRIM) protein family. TRIM21 is an E3 ubiquitin ligase that can specifically recognize and bind to target proteins, thereby promoting their ubiquitination and ultimately mediating their degradation in the proteasome. The TRIM-binder technology combines the high specificity of binders with the efficient degradation activity of TRIM21, providing a more efficient and precise strategy for PCNA degradation that is expected to enhance the therapeutic effect for PCNA-related cancers.

However, to date, no research has been reported on PCNA-targeting binders.

### SUMMARY

The present disclosure provides a binder that specifically targets the PCNA protein, a fusion protein comprising the same, and uses thereof, which address the problem in the prior art of lacking binders with good specificity and high affinity that are capable of efficiently binding to the PCNA antigen.

In a first aspect, the present disclosure provides a binder specifically targeting proliferating cell nuclear antigen (PCNA), wherein the binder is selected from any one of the following:
A1) an amino acid sequence as shown in any one of SEQ ID NOs: 1-6;
A2) an amino acid sequence having one or several amino acid substitutions, deletions, or additions compared to the amino acid sequence defined in A1);
A3) an amino acid sequence having at least 80% sequence identity to the amino acid sequence defined in A1) or A2);
A4) an amino acid sequence obtained by linking a tag or a signal peptide to the N-terminus and/or C-terminus of the amino acid sequence defined in A1), A2) or A3).

In the present disclosure, binders that specifically target and bind to PCNA were designed via artificial intelligence technology. Said binders exhibit good specificity and high affinity, and are capable of efficiently binding to the PCNA antigen.

In a second aspect, the present disclosure provides a fusion protein, which is prepared by fusing any one of the aforementioned binders to an RBCC domain or its mutant via a third linker, or by fusing the aforementioned binders directly to an Fc fragment; wherein the amino acid sequence of the third linker is shown in SEQ ID NO: 18, the amino acid sequence of the RBCC domain is shown in SEQ ID NO: 16, and the amino acid sequence of the RBCC domain mutant is shown in SEQ ID NO: 17.

In the present disclosure, the aforementioned binders with high affinity and good specificity are fused to an RBCC domain or a mutant thereof (a truncated TRIM family protein), or directly fused to an Fc fragment, to obtain fusion proteins. The resulting fusion proteins can specifically bind to a target protein via their binder domains, thereby initiating a protein degradation pathway to achieve targeted degradation of the PCNA protein. Meanwhile, said fusion proteins can also activate intracellular p53 and induce its phosphorylation to prevent p53 from undergoing ubiquitination and degradation by ubiquitin ligases such as MDM2, thereby promoting apoptosis and inhibiting cancer cell proliferation. The binders and fusion proteins of the present disclosure exhibit significant clinical application value.

It will be appreciated that linker peptides used in the fusion process may be selected from conventional linkers known in the art according to practical needs, provided that they allow for sufficient fusion of the two proteins.

In some embodiments, the fusion protein is selected from any one of the following:
B1) an amino acid sequence as shown in any one of SEQ ID NOs: 22-31;
B2) an amino acid sequence having one or several amino acid substitutions, deletions or additions compared to the amino acid sequence defined in B1);
B3) an amino acid sequence having at least 80% sequence identity compared to the amino acid sequence defined in B1) or B2);
B4) an amino acid sequence obtained by linking a tag or a signal peptide to the N-terminus and/or C-terminus of the amino acid sequence defined in B1), B2) or B3).

The aforementioned binders and fusion proteins may be active polypeptides that are natural, recombinant, or synthetic. Such active polypeptides may be naturally purified products, chemically synthesized products, or products produced from a prokaryotic host (e.g., Escherichia coli) or a eukaryotic host (e.g., yeast, higher plant) using recombinant technology.

In the present disclosure, in A4) and B4), the linking may be performed either directly via a peptide bond or via a linker, using methods conventional in the art. Tags include, but are not limited to: GST (glutathione S-transferase) tag, Trx (thioredoxin) tag, His tag (His-tag), Flag tag, LacZ tag, GFP (green fluorescent protein), sfGFP (superfolder green fluorescent protein), and HA tag (hemagglutinin tag). Those skilled in the art may select an appropriate tag according to practical needs. Use of the tag does not alter the function of the target protein (binder or fusion protein), as it is intended for isolation, purification, detection, or tracing. The tag may be cleaved from the target protein (binder or fusion protein) by chemical cleavage or enzymatic methods known in the art (e.g., introducing a protease cleavage site to enable tag removal by cleavage with TEV protease).

In a third aspect, the present disclosure provides a nucleic acid molecule encoding any one of the aforementioned binders or any one of the aforementioned fusion proteins.

In some embodiments, the nucleic acid molecule is selected from any one of the following:
C1) a nucleic acid molecule having a nucleotide sequence as shown in any one of SEQ ID NOs: 7-12;
C2) a nucleic acid molecule that hybridizes to the nucleic acid molecule defined in C1) under stringent conditions and encodes any one of the aforementioned binders;
C3) a nucleic acid molecule having at least 90% sequence identity to the nucleic acid molecule defined in C1) or C2) and encoding any one of the aforementioned binders.

The nucleic acid molecules provided herein may be DNA, such as cDNA, genomic DNA, or recombinant DNA; or RNA, such as mRNA or hnRNA. Such nucleic acid molecules may generally be obtained by PCR amplification or artificial synthesis.

In a fourth aspect, the present disclosure provides a recombinant vector comprising any one of the aforementioned nucleic acid molecules.

The recombinant vectors provided herein include cloning vectors and expression vectors. Cloning vectors are used for replicating related sequences, and expression vectors are used for expressing related genes. The vector used to construct the expression vector can be at least one of pET23a and pcDNA3.1.

In a fifth aspect, the present disclosure provides a recombinant cell comprising any one of the aforementioned nucleic acid molecules or the aforementioned recombinant vectors.

In some embodiments, the method for preparing the aforementioned recombinant cell comprises the step of transforming the aforementioned recombinant vector into an expression host cell.

In the present disclosure, the expression host cell is a conventional host cell in the art, so long as it allows stable self-replication of the recombinant vector and effective expression of the genes carried therein. It may be a prokaryotic cell or a eukaryotic cell, such as Escherichia coli (E. coli) or yeast. Examples of E. coli include E. coli BL21(DE3), Rosetta(DE3), and BL21(DE3)plysS. In the present disclosure, E. coli BL21(DE3) is preferred.

In a sixth aspect, the present disclosure provides use of any one of the aforementioned binders, any one of the aforementioned fusion proteins, any one of the aforementioned nucleic acid molecules, the aforementioned recombinant vector, or the aforementioned recombinant cell in any one of the following:
D1) use in the preparation of a product for the prophylaxis and/or treatment of a PCNA target-related disease;
D2) use in the preparation of a product for the screening, diagnosis or auxiliary diagnosis of a PCNA target-related disease;
wherein the PCNA target-related disease is a PCNA-positive tumor.

In the present disclosure, “PCNA-positive tumor” refers to a tumor in which PCNA expression is detected in tumor cells. Examples of such tumors include common cancers such as lung cancer, gastric cancer, liver cancer, breast cancer, prostate cancer, colorectal cancer, and esophageal cancer.

In a seventh aspect, the present disclosure provides a pharmaceutical composition for the prophylaxis and/or treatment of a PCNA target-related disease, which comprises any one of the aforementioned binders, any one of the aforementioned fusion proteins, any one of the aforementioned nucleic acid molecules, the aforementioned recombinant vector, or the aforementioned recombinant cell, and a pharmaceutically acceptable carrier.

In the present disclosure, the term “pharmaceutically acceptable carrier” refers to an excipient widely used in the field of pharmaceutical manufacturing. Excipients are primarily used to provide a safe, stable, and functional pharmaceutical composition. They may also enable a desired dissolution rate of the active ingredient after administration to a subject, or facilitate effective absorption of the active ingredient by the subject after administration of the composition. The pharmaceutical excipient may be an inert filler, or may provide a certain function, such as stabilizing the overall pH of the composition or preventing degradation of the active ingredients of the composition.

The pharmaceutical composition provided herein can be prepared by any method known to those skilled in the art based on the present disclosure. Such methods include, but are not limited to, conventional mixing, dissolving, granulating, emulsifying, comminuting, encapsulating, embedding or lyophilizing processes.

In some embodiments, the dosage form of the pharmaceutical composition includes at least one of a solid formulation, a semi-solid formulation, and a liquid formulation.

The pharmaceutical composition provided herein may be administered in any form, including injection (intravenous), mucosal, oral (solid and liquid preparations), inhalation, ocular, rectal, topical or parenteral (infusion, injection, implantation, subcutaneous, intravenous, intraarterial, intramuscular) administration. The pharmaceutical composition of the present disclosure may also be in a controlled-release or sustained-release dosage form (e.g., a liposome or microsphere).

In an eighth aspect, the present disclosure provides a method for preparing any one of the aforementioned binders or any one of the aforementioned fusion proteins, comprising the steps of:
culturing the aforementioned recombinant cell and inducing expression thereof to obtain a culture; and
isolating the binder or the fusion protein from the culture.

In the present disclosure, there are no particular limitations on the culture method, culture conditions, and culture medium, so long as the recombinant cell can grow normally. Methods for isolating any one of the aforementioned binders or fusion proteins from the culture are all conventional methods in the art.

The beneficial effects of the present disclosure are as follows:

Different from the prior art, the present disclosure employs artificial intelligence technology to design binders specifically targeting PCNA. The resulting binders exhibit good specificity and high affinity, and are capable of efficiently binding to the PCNA antigen. Further, fusion proteins are prepared by fusing the aforementioned binders to an RBCC domain or a mutant thereof, or by directly fusing the aforementioned binders to an Fc fragment. These fusion proteins can specifically bind to the target protein via their binder domain, thereby initiating a protein degradation pathway to achieve targeted degradation of the PCNA protein, and they can also activate intracellular p53, inducing its phosphorylation to prevent ubiquitination-mediated degradation by ubiquitin ligases such as MDM2, thus giving the present disclosure substantial clinical application value.

### BRIEF DESCRIPTION OF DRAWINGS

FIG. 1 is a schematic diagram showing the element arrangement of the recombinant expression vectors constructed in the present disclosure;

FIG. 2 shows the SDS-PAGE analysis results of the binders in Example 2 of the present disclosure, wherein Lane M is Marker, Lanes 1-6 are PB1-sfGFP, PB2-sfGFP, PB3-sfGFP, PB4-sfGFP, PB5-sfGFP and PB6-sfGFP respectively, and Lanes 7-8 are PB25-sfGFP and PB52-sfGFP respectively;

FIG. 3 shows the SDS-PAGE analysis results of the PCNA antigen in Example 2 of the present disclosure;

FIGS. 4-5 show the ELISA results of the affinity between the binders and the PCNA antigen in Example 3 of the present disclosure;

FIG. 6 shows the BLI results of the affinity between the binders and the PCNA antigen in Example 4 of the present disclosure, wherein (a) and (b) are structural simulation diagrams, and (c) is an affinity result diagram;

FIG. 7 shows the Western blot (WB) results of targeted PCNA protein degradation by the fusion proteins (RBCC-PB2 and RBCC-PB25) in Example 5 of the present disclosure, wherein (a) is a gel analysis diagram, and (b) is a relative expression diagram of the PCNA antigen;

FIG. 8 shows the Western blot (WB) results of targeted PCNA protein degradation by the fusion protein RS79E-PB2 (a) and the phosphorylation level results of p53 (b) in Example 5 of the present disclosure;

FIG. 9 shows the Western blot (WB) results of targeted PCNA protein degradation by the fusion proteins (PB127-Fc and PB127-GFP-Fc) in Example 5 of the present disclosure;

FIG. 10 shows the Western blot results verifying the pathway of targeted PCNA protein degradation by fusion proteins in Example 5 of the present disclosure.

### DETAILED DESCRIPTION OF THE EMBODIMENTS

The technical solutions in the embodiments of the present disclosure will be clearly and fully described below in conjunction with the accompanying examples. It will be apparent that the embodiments described are merely some, rather than all, embodiments of the present disclosure. Other embodiments obtained by those of ordinary skill in the art without creative efforts based on the embodiments of the present disclosure shall fall within the scope of protection of the present disclosure.

Experimental methods for which specific conditions are not indicated in the following examples are usually performed according to conventional experimental methods in the field of molecular biology, including, but not limited to, those described in Molecular Cloning: A Laboratory Manual by M. R. Green, Molecular Biology by Robert F. Weaver, or as recommended by the manufacturers of kits and instruments. Reagents and biological materials used in the examples are commercially available unless otherwise specified.

### Example 1: Using Artificial Intelligence to Design Binders

First, artificial intelligence technology in combination with tools including ProteinMPNN, RFdiffusion, AlphaFold3, and HADDOCK was employed to design a series of binders targeting specific epitopes of PCNA, which are respectively designated as binders PB1-6, PB25, PB52, PB127, and PB127-GFP.

Exemplarily, the amino acid sequence of binder PB2 is shown below:

(SEQ ID NO: 1)
MKKYVIEEELSDEELLEKFKEIVELMKEEPVDITVTGAHALEIFRAVTEL
AKLLGVKVNIDVTLTESSAKYEPLEKKMLEYVRETVEAEKKKEELA;

Exemplarily, the amino acid sequence of binder PB5 is shown below:

(SEQ ID NO: 2)
KSVVYELTYEDKSGNKVKVTVTYDEETGEITLTQERTSKRVVNGKLVEV
KETFLWRLSSSGTAEEALATIKKAYQRGAKTPVKVSFVKEIS;

Exemplarily, the amino acid sequence of binder PB6 is shown below:

(SEQ ID NO: 3)
MMKNVDVDIKYDKDGTIKVTIKSEDTTIEIEEKVKTKEEREAVLDIVFEV
MRRLLKGEDIEEIKKKMEEELKKIK;

Exemplarily, the amino acid sequence of binder PB25 is shown below:

(SEQ ID NO: 4)
MKKYVIEEELSDEELLEKFKEIVELMKEEPVDITVTGAHALEIFRAVTEL
AKLLGVKVNIDVTLTESSAKYEPLEKKMLEYVRETVEAEKKKEELAGGGG
SGGGGSGGGGSKSVVYELTYEDKSGNKVKVTVTYDEETGEITLTQERTSK
RVVNGKLVEVKETFLWRLSSSGTAEEALATIKKAYQRGAKTPVKVSFVKE
IS;

Exemplarily, the amino acid sequence of binder PB52 is shown below:

(SEQ ID NO: 5)
KSVVYELTYEDKSGNKVKVTVTYDEETGEITLTQERTSKRVVNGKLVEVK
ETFLWRLSSSGTAEEALATIKKAYQRGAKTPVKVSFVKEISGGGGSGGGG
SGGGGSMKKYVIEEELSDEELLEKFKEIVELMKEEPVDITVTGAHALEIF
RAVTELAKLLGVKVNIDVTLTESSAKYEPLEKKMLEYVRETVEAEKKKEE
LA;

Exemplarily, the amino acid sequence of binder PB127 is shown below:

(SEQ ID NO: 6)
SEKEEELKKLIEEAIKLEKEGKKEEAKKKLEEALELAKELGYDATAEAVQ
QKLDKL.

The nucleotide sequences of the genes encoding binders PB2, PB5, PB6, PB25, PB52, and PB127 are shown in SEQ ID NOs: 7-12 respectively.

### Example 2: Expression and Purification of Binders and PCNA Antigen

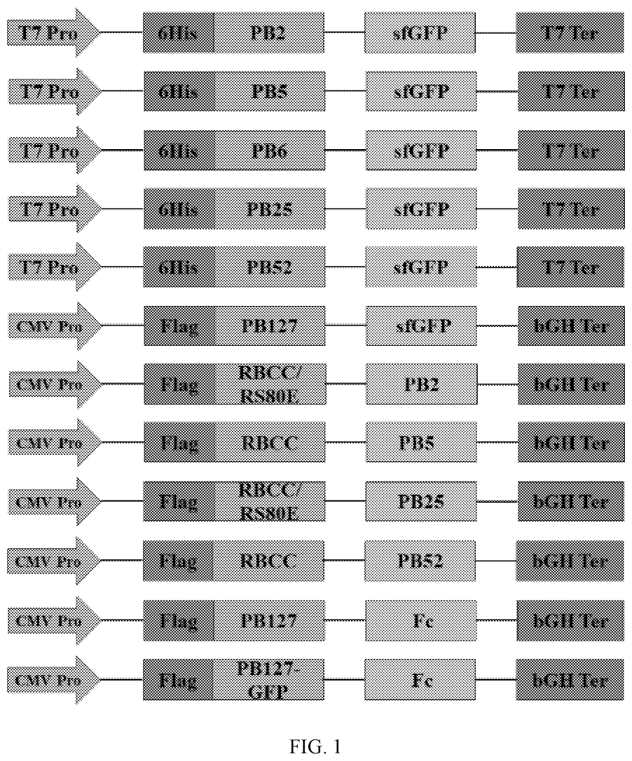

To facilitate the isolation and purification of binders, in the present disclosure, the binders of Example 1 were displayed on the surface of Escherichia coli via superfolder green fluorescent protein (sfGFP), wherein the nucleotide sequence of the gene encoding sfGFP protein is shown in SEQ ID NO: 13. Specifically, using pET23a as the backbone vector, the genes encoding the aforementioned binders PB2, PB5, PB6, and PB127 were fused with the sfGFP gene via a first linker (the nucleotide sequence of which is shown in SEQ ID NO: 14), and the genes encoding the aforementioned binders PB25 and PB52 were fused with the sfGFP gene via a second linker (the nucleotide sequence of which is shown in SEQ ID NO: 15). Expression vectors were constructed by homologous recombination. The element arrangement in these expression vectors is shown in FIG. 1. Exemplarily, the recombinant plasmids were pET23a-PB2-sfGFP, pET23a-PB5-sfGFP, pET23a-PB6-sfGFP, pET23a-PB25-sfGFP, pET23a-PB52-sfGFP, and pET23a-PB127-sfGFP, respectively.

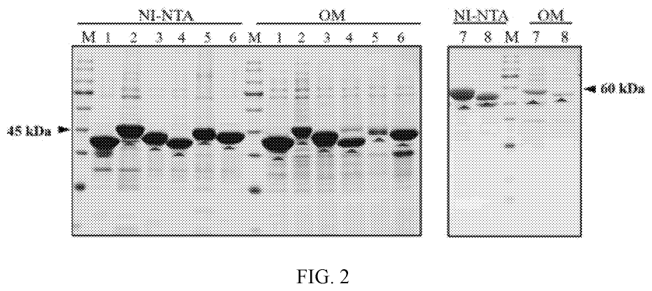

The recombinant plasmids constructed above were individually transformed into E. coli competent cells BL21(DE3), followed by static cultivation at 37° C. overnight to obtain a series of recombinant strains. Single colonies were then picked and inoculated into 100 mL of LB liquid medium (containing 50 μg/mL ampicillin) for shake cultivation at 37° C. When the OD₆₀₀ reached approximately 0.6, isopropyl β-D-1-thiogalactopyranoside (IPTG) was added to a final concentration of 0.5 mM, and induction was performed by shaking cultivation at 18° C. for 18 hours. After cultivation, the bacterial cells and the culture supernatant were separately collected by centrifugation at 12,000 rpm and 4° C. The bacterial cells were washed with TEN buffer (50 mM Tris-HCl, 5 mM EDTA, 50 mM NaCl, pH 8.0) at 4° C. for more than 4 hours, and the proteins secreted and expressed by the strains were directly obtained from the TEN buffer. Sodium dodecyl sulfate-polyacrylamide gel electrophoresis (SDS-PAGE) was performed to detect and analyze the secretory expression of the binders. The results are shown in FIG. 2.

As can be seen from FIG. 2, binders PB1-6, PB25, and PB52 were all successfully expressed.

Representatively, the purified binders PB1-6, PB25, PB52 and PB127-GFP obtained by outer membrane washing were stored for subsequent experiments.

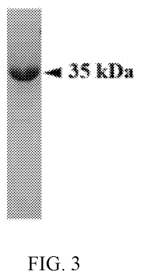

Further, the PCNA antigen sequence (adopting amino acids 1-261 of NCBI Accession No.: P12004) was cloned into the pET23a vector by homologous recombination to obtain the recombinant plasmid pET23a-PCNA. The constructed recombinant plasmid was transformed into E. coli competent cells BL21(DE3). A single colony was picked and inoculated into 100 mL of LB liquid medium (containing 50 μg/mL ampicillin) for static cultivation at 37° C. overnight. When the OD₆₀₀ reached approximately 0.6, IPTG was added to a final concentration of 0.5 mM, and induction was performed by shaking cultivation at 18° C. for 18 hours. The bacterial cells were collected by centrifugation at 6,000 rpm for 10 min, washed with phosphate-buffered saline (PBS), and then resuspended in PBS. Phenylmethylsulfonyl fluoride (PMSF) was added to a final concentration of 1 mM, followed by high-pressure disruption of the cells. The resulting lysate was centrifuged at 18,000 rpm for 30 min, and the supernatant was collected, filtered, and purified by Ni-NTA affinity chromatography. The purified protein was collected and detected by SDS-PAGE. It was then aliquoted, snap-frozen in liquid nitrogen, and stored at −80° C. The identification and analysis results of SDS-PAGE are shown in FIG. 3. The predicted molecular weight of PCNA was calculated to be 29.8 kDa via http://www.expasy.org/, and the results indicated that the PCNA antigen was successfully expressed.

### Example 3: ELISA Assay of Binders

This example aims to verify whether the purified binders PB1-6 and PB127-GFP from Example 2 can directly interact with the purified PCNA antigen from Example 2. The detailed steps are as follows:
a) Diluting the PCNA antigen to 1 μg/mL with 1×ELISA coating buffer, adding the diluted antigen solution to a microplate at 100 μL/well, and incubating the microplate statically at 4° C. overnight;
b) Washing the microplate with PBST, and blocking the microplate with 1% BSA at room temperature for 2 h at 100 μL/well;
c) Preparing binders PB1-6 and PB127-GFP at various concentrations with 1% BSA, adding the binder solution to the microplate at 100 μL/well, and incubating the microplate at room temperature for 1 h;
d) Incubating with HRP-conjugated Mouse anti-HA-Tag mAb (secondary antibody) at room temperature for 1 h;
e) Developing color with TMB substrate solution, and then terminating the reaction with stop solution;
f) Measuring the absorbance at 450 nm using a microplate reader.

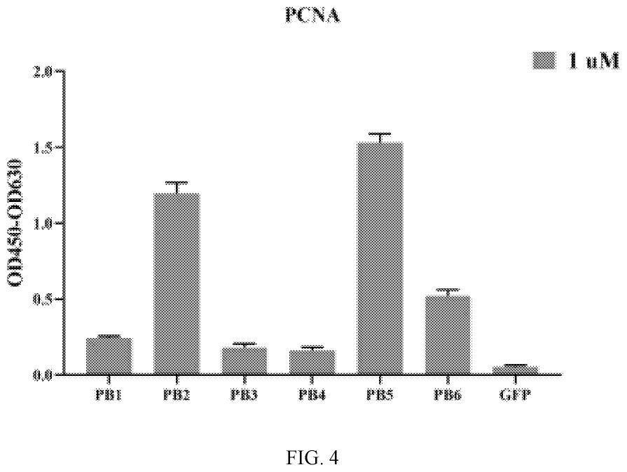

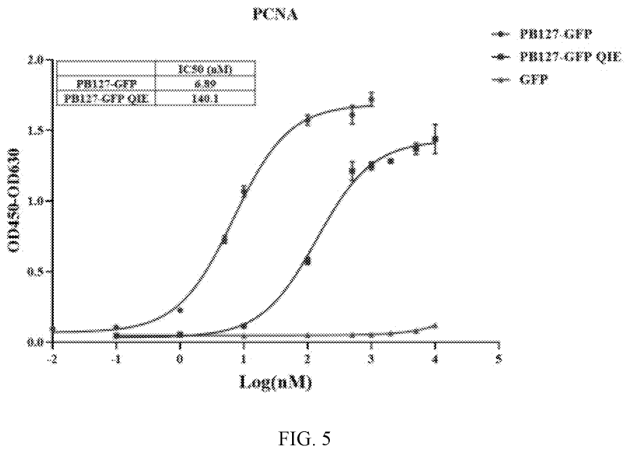

The test results are shown in FIGS. 4 and 5, respectively.

It can be seen from FIGS. 4 and 5 that binders PB2, PB5, PB6, and PB127-GFP have good affinity for the PCNA antigen, and the affinity of PB127-GFP for PCNA is approximately 20 times higher than that of PB127.

### Example 4: Biolayer Interferometry (BLI) Assay of Binders

This example aims to further verify the binding between the purified binders PB2, PB5, PB25, and PB52 from Example 2 and the purified PCNA antigen from Example 2, and to calculate the affinity between them.

Specifically, the PCNA antigen was immobilized onto a biosensor chip, and the binders PB2, PB5, PB25, and PB52 at different concentrations were separately added to analyze their affinity for the antigen protein. The increase in optical thickness at the tip of the biosensor was recorded over 600 seconds. The affinity for PCNA was calculated according to the association and dissociation rates of the binders at different concentrations.

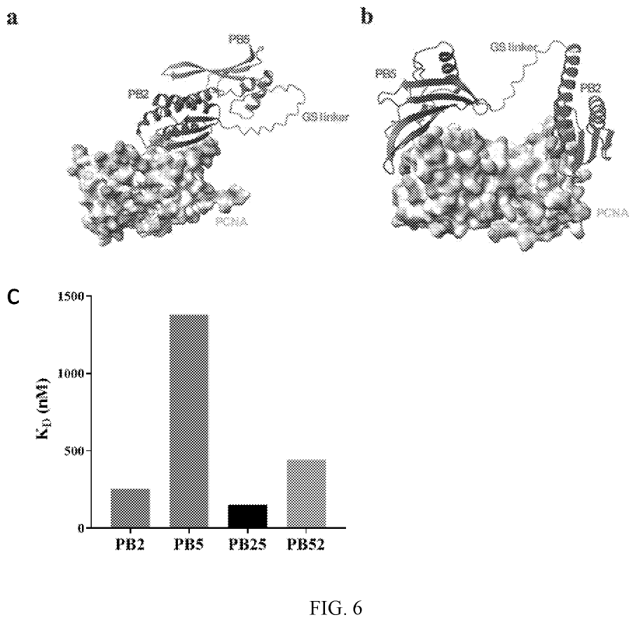

The results are shown in FIG. 6. As can be seen from FIG. 6, binders PB2, PB5, PB25, and PB52 exhibit favorable affinity for the PCNA antigen, and binder PB25 shows an even better affinity for the PCNA antigen.

### Example 5: Targeted Degradation of the PCNA Protein by Fusion Proteins

To achieve targeted degradation of PCNA protein, in the present disclosure, the aforementioned binders PB2, PB5, PB6, PB25, PB52, and PB127 were respectively fused with the RBCC domain or its mutant RS79E to obtain fusion proteins. The fusion proteins were expressed in HEK293T cells, and the targeted degradation efficiency of PCNA protein by these fusion proteins was detected.

Specifically, using pcDNA3.1 as the backbone vector, the genes encoding the aforementioned binders PB2, PB5, PB6, PB25, PB52, and PB127 were fused with the gene encoding the RBCC domain (the amino acid sequence of which is shown in SEQ ID NO: 16, and the nucleotide sequence of which is shown in SEQ ID NO: 19) or its mutant RS79E (the amino acid sequence of which is shown in SEQ ID NO: 17, and the nucleotide sequence of which is shown in SEQ ID NO: 20) via a third linker (the amino acid sequence of which is shown in SEQ ID NO: 18, and the nucleotide sequence of which is shown in SEQ ID NO: 21). Expression vectors were constructed by homologous recombination. The element arrangement in these expression vectors is shown in FIG. 1. The recombinant plasmids were pcDNA3.1-RBCC-PB2, pcDNA3.1-RBCC-PB5, pcDNA3.1-RBCC-PB6, pcDNA3.1-RBCC-PB25, pcDNA3.1-RBCC-PB52, pcDNA3.1-RBCC-PB127, pcDNA3.1-RS79E-PB2, pcDNA3.1-RS79E-PB25, pcDNA3.1-PB127-Fc, and pcDNA3.1-PB127-GFP-Fc, respectively.

The amino acid sequences of the fusion proteins RBCC-PB2, RBCC-PB5, RBCC-PB6, RBCC-PB25, RBCC-PB52, RBCC-PB127, RS79E-PB2, RS79E-PB25, PB127-Fc, and PB127-GFP-Fc are shown in SEQ ID NOs. 22-31, respectively.

The nucleotide sequences of the genes encoding the fusion proteins PB127-Fc and PB127-GFP-Fc are shown in SEQ ID NOs: 32-33, respectively.

Exemplarily, transfection was performed using PEI reagent. The recombinant plasmids pcDNA3.1-RBCC-PB2, pcDNA3.1-RBCC-PB25, and the GFP plasmid were electrotransformed into well-growing HEK293T cells, respectively. After electrotransformation, the transfection efficiency and protein expression were monitored using the GFP signal.

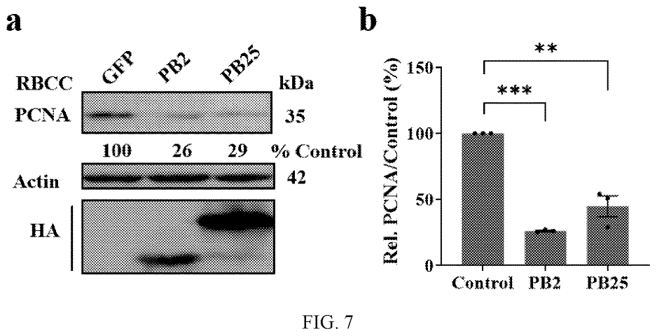

Cells were harvested 48 h post-transfection, and then subjected to Western blot (WB) analysis. Based on the WB results, the targeted protein degradation efficiency at 48 h post-transfection was calculated. The results are shown in FIG. 7.

As can be seen from FIG. 7, the fusion protein RBCC-PB2 exhibits the most significant degradation effect on the PCNA antigen, with the targeted protein degradation efficiency of about 70% at 48 h.

According to the method described above, exemplarily, the recombinant plasmid pcDNA3.1-RS79E-PB2 and the GFP plasmid were electrotransformed into well-growing HEK293T cells, respectively. After electrotransformation, the transfection efficiency and protein expression were monitored using the GFP signal.

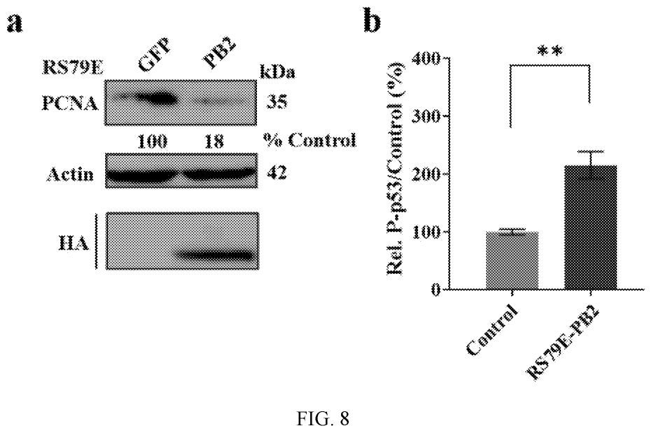

Cells were harvested 48 h post-transfection, and then subjected to WB analysis. Based on the WB results, the targeted protein degradation efficiency at 48 h post-transfection was calculated. The results are shown in FIG. 8.

As can be seen from FIG. 8, the fusion protein RS79E-PB2 also exhibits a significant degradation effect on the PCNA antigen, and the targeted degradation efficiency of the protein is about 80% at 48 h. Moreover, compared with the control group, the level of phosphorylated p53 is significantly increased after targeted degradation of PCNA by RS79E-PB2 in the experimental group.

Similarly, according to the method described above, exemplarily, the recombinant plasmids pcDNA3.1-PB127-Fc, pcDNA3.1-PB127-GFP-Fc, the Trim21 plasmid, and the GFP plasmid were electrotransformed into well-growing HEK293T cells, respectively. After electrotransformation, the transfection efficiency and protein expression were monitored using the GFP signal.

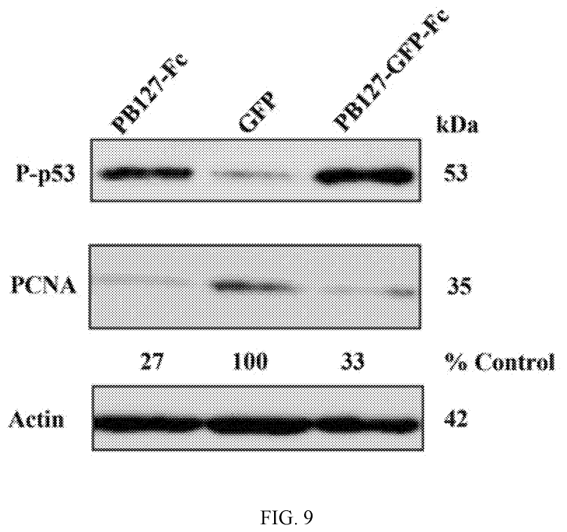

Cells were harvested 48 h post-transfection, and then subjected to WB analysis. Based on the WB results, the targeted protein degradation efficiency at 48 h post-transfection was calculated. The results are shown in FIG. 9.

As can be seen from FIG. 9, both plasmids pcDNA3.1-PB127-Fc and pcDNA3.1-PB127-GFP-Fc exert significant targeted protein degradation effects after electrotransformation.

Finally, the degradation pathway by which the fusion proteins targetedly degrade the PCNA protein was investigated.

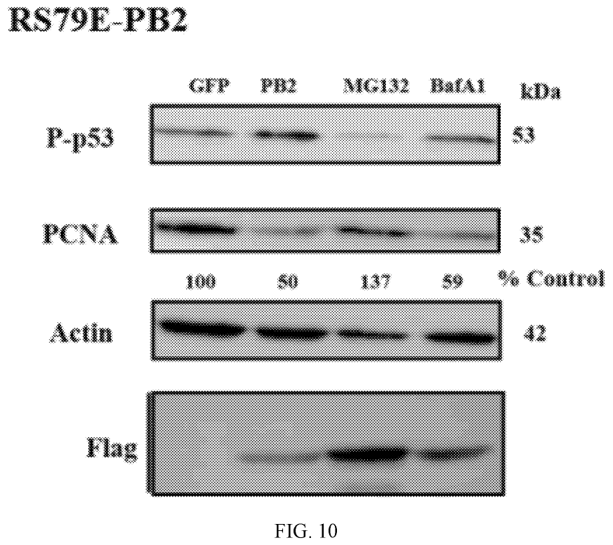

Specifically, MG132 proteasome inhibitor at a final concentration of 15 μM or BafA1 autophagy inhibitor at a final concentration of 200 nM was used, in which MG132 can inhibit the ubiquitin-proteasome pathway and BafA1 can inhibit the lysosomal pathway. Three groups of well-growing HEK293T cells were electrotransformed with pcDNA3.1-RS79E-PB2 recombinant plasmid and GFP plasmid. At 24 h post-transfection, MG132 and BafA1 were added to two of the groups of cells carrying the pcDNA3.1-RS79E-PB2 recombinant plasmid, respectively. After further culture, WB analysis was performed. The results are shown in FIG. 10.

As can be seen from FIG. 10, the targeted degradation of the PCNA protein by the fusion proteins in the present disclosure is achieved through the ubiquitin-proteasome pathway.

In summary, the present disclosure utilizes artificial intelligence technology to design binders that specifically target PCNA. The resulting binders exhibit good specificity and high affinity, and are capable of efficiently binding to the PCNA antigen. Further, fusion proteins are prepared by fusing the aforementioned binders to an RBCC domain or a mutant thereof, or by directly fusing the aforementioned binders to an Fc fragment. These fusion proteins can specifically bind to the target protein via their binder domain, activate the protein degradation pathway, and achieve targeted degradation of PCNA protein.

The foregoing examples merely represent certain embodiments of the present disclosure. The descriptions thereof are relatively specific and detailed, but should not be construed as limiting the scope of the the present disclosure. It should be noted that, for those of ordinary skill in the art, any modifications, equivalent replacements and improvements made within the concepts and principles of the present disclosure shall fall within the protection scope of the present disclosure. Therefore, the scope of protection of the present disclosure shall be subject to the appended claims.

What is claimed is:

1. A binder targeting PCNA protein, wherein the binder is selected from any one of A1) and A2):
A1) an amino acid sequence as shown in any one of SEQ ID NOs: 1, 4, and 5;
A2) an amino acid sequence obtained by linking a tag or a signal peptide to the N-terminus and/or C-terminus of the amino acid sequence defined in A1).

2. A fusion protein, wherein the fusion protein is prepared by fusing the binder of claim 1 to an RBCC domain or its mutant via a third linker;
wherein the amino acid sequence of the third linker is shown in SEQ ID NO: 18, the amino acid sequence of the RBCC domain is shown in SEQ ID NO: 16, the amino acid sequence of the mutant of the RBCC domain is shown in SEQ ID NO: 17; and the amino acid sequence of the fusion protein is shown in any one of SEQ ID NOs: 22, 25, and 28.

3. A nucleic acid molecule encoding the binder of claim 1 or a fusion protein thereof;
wherein the fusion protein is prepared by fusing the binder to an RBCC domain or its mutant via a third linker, the amino acid sequence of the third linker is shown in SEQ ID NO: 18, the amino acid sequence of the RBCC domain is shown in SEQ ID NO: 16, the amino acid sequence of the mutant of the RBCC domain is shown in SEQ ID NO: 17; and the amino acid sequence of the fusion protein is shown in any one of SEQ ID NOs: 22, 25, and 28.

4. The nucleic acid molecule of claim 3, wherein the nucleic acid molecule has a nucleotide sequence as shown in any one of SEQ ID NOs: 7, 10, and 11.

5. A recombinant vector comprising the nucleic acid molecule of claim 3.

6. The recombinant vector of claim 5, wherein the nucleic acid molecule has a nucleotide sequence as shown in any one of SEQ ID NOs: 7, 10, and 11.

7. A recombinant cell comprising the nucleic acid molecule of claim 3 or a recombinant vector that comprises said nucleic acid molecule.

8. The recombinant cell of claim 7, wherein the nucleic acid molecule has a nucleotide sequence as shown in any one of SEQ ID NOs: 7, 10, and 11.

9. A pharmaceutical composition, comprising any one selected from E1) to E5), and a pharmaceutically acceptable carrier;
E1) the binder of claim 1;
E2) a fusion protein prepared by fusing the binder of E1) to an RBCC domain or its mutant via a third linker; wherein the amino acid sequence of the third linker is shown in SEQ ID NO: 18, the amino acid sequence of the RBCC domain is shown in SEQ ID NO: 16, the amino acid sequence of the mutant of the RBCC domain is shown in SEQ ID NO: 17; and the amino acid sequence of the fusion protein is shown in any one of SEQ ID NOs: 22, 25, and 28;
E3) a nucleic acid molecule encoding the binder of E1) or the fusion protein of E2);
E4) a recombinant vector comprising the nucleic acid molecule of E3);
E5) a recombinant cell comprising the nucleic acid molecule of E3) or the recombinant vector of E4).

10. The pharmaceutical composition of claim 9, wherein the nucleic acid molecule has a nucleotide sequence as shown in any one of SEQ ID NOs: 7, 10, and 11.

11. A method for preparing the binder of claim 1 or a fusion protein thereof, comprising the steps of:
culturing a recombinant cell and inducing expression thereof to obtain a culture; and
isolating the binder or the fusion protein from the culture;
wherein the recombinant cell comprises a nucleic acid molecule encoding the binder or the fusion protein, or the recombinant cell comprises a recombinant vector that comprises said nucleic acid molecule
wherein the fusion protein is prepared by fusing the binder of claim 1 to an RBCC domain or a mutant thereof via a third linker, the amino acid sequence of the third linker is shown in SEQ ID NO: 18, the amino acid sequence of the RBCC domain is shown in SEQ ID NO: 16, the amino acid sequence of the mutant of the RBCC domain is shown in SEQ ID NO: 17; and the amino acid sequence of the fusion protein is shown in any one of SEQ ID NOs: 22, 25, and 28.

12. The method of claim 11, wherein the nucleic acid molecule has a nucleotide sequence as shown in any one of SEQ ID NOs: 7, 10, and 11.
